(19) United States
(12) Patent Application Publication
(10) Pub. No.: US 2003/0113758 A1
(43) Pub. Date: Jun. 19, 2003

(54) Method for the in vitro diagnosis of a predisposition to bladder cancer or of the occurrence of bladder cancer and a kit for performing said diagnostic method

(76) Inventors: Pierre Oudet, Strasbourg (FR); Didier Jacqmin, Lingolsheim (FR); Marie-Pierre Gaub, Strasbourg (FR); Anne Schneider, Strasbourg (FR); Herve Lang, Strasbourg (FR)

Correspondence Address: YOUNG & THOMPSON, 745 SOUTH 23RD STREET 2ND FLOOR, ARLINGTON, VA, 22202

(21) Appl. No.: 10/217,464

(22) Filed: Aug. 14, 2002

Related U.S. Application Data
(60) Provisional application No. 60/311,846, filed on Aug. 14, 2001.

(51) Int. Cl. C12Q 1/68

(52) U.S. Cl. 435/6

(57) ABSTRACT

A new in vitro method for diagnosing the predisposition of a human individual to bladder cancer or for diagnosing the occurrence of a bladder cancer in a human individual, makes use of a comparison between the allelic ratios of a serial of microsatellite markers associated with this disease, respectively in the urine DNA and in the blood cell DNA of said human individual.

### FIELD OF THE INVENTION

The present invention relates to a new in vitro method for diagnosing the predisposition of a human individual to bladder cancer or for diagnosing the occurrence of a bladder cancer in a human individual, wherein said method makes use of a comparison between the allelic ratios of a serial of microsatellite markers associated with this disease, respectively in the urine DNA and in the blood cell DNA of said human individual.

It is also directed to diagnostic kits which are useful for carrying out the method above.

Throughout this Application, various publications are cited. The disclosures of these publications referenced in this Application are hereby incorporated by reference into the present disclosure to more fully describe the state of the art to which this invention pertains.

### BACKGROUND OF THE INVENTION

Bladder cancer is the fourth cancer in men and the eighth in women both in terms of incidence and mortality (1-3). The main risk of these tumors is a high frequency of recurrence and progression depending on their initial stage and grade (4-6).

Diagnosis of bladder cancer at an early stage appears to be one of the main factors for patients survival. Cystoscopy is the “gold standard” method for diagnosis and follow-up but is still invasive for the patients. Cytology is a common non-invasive procedure for diagnosis but can miss up to 50% of tumors especially those of low grade and low stage (5, 7). Therefore there is a need for a diagnosis method less invasive than cystoscopy and more efficient than cytology which could also be used for the follow-up of bladder tumors.

Genomic rearrangements are very often observed in tumors and their accumulation is a sign of cancer progression. In bladder transitional cell carcinoma (TCC), several studies have shown recurrent loss of heterozygosity at chromosomes 3, 4, 8, 9, 11, 13, 17 and 18 involving tumor suppressor genes such as p53 and p16 (8-16). Furthermore, chromosome 9 alterations appeared to occur early in bladder carcinogenesis (15). Recently, microsatellite analysis was shown to detect allelic imbalance and genomic instability in primary tumors (17-21). Genomic or microsatellite instability describes accumulation of modifications in number of repeats due to failure of DNA mismatch repair mechanism (22-24). Allelic imbalance refers to partial or complete loss of one of the two alleles (previously known as loss of heterozygosity) or alternatively amplification of one allele compared to the other (25, 26).

Recent studies have detected identical microsatellite alterations in bladder tumor and corresponding urine sediment from the same patient, demonstrating the ability to identify clonal population of tumor-derived cells in urine sediment (17, 27).

Despite the fact that some prior art academic studies have shown that an accumulation of DNA mutations occurred in patients suffering from bladder cancer, and that some of these mutations can be detected in tumor-derived urine cells, no reproducible nor reliable method which can be safely and routinely used at a diagnostic industrial scale was, to date, available nor could be easily derived from the prior art knowledge.

However, there exists a great need in the art for such a diagnostic method which should be also highly sensitive and highly specific for bladder cancer diganosis.

### SUMMARY OF THE INVENTION

A first object of the invention consists of a method for the in vitro diagnosis of a predisposition to bladder cancer or of the occurrence of a bladder cancer in a human patient, wherein said method comprises the steps of:

a) performing an allelotyping analysis respectively (i) of the blood DNA and (ii) of the urine DNA from said patient, using a serial of microsatellite markers associated with bladder cancer,

b) determining the allele ratio for each microsatellite marker respectively (i) (Bb/Ba) for the blood DNA and (ii) (Ub/Ua) for the urine DNA, wherein:

Ba and Bb represent the respective frequency of the two alleles of said microsatellite marker in the blood DNA of said patient, and

Ua and Ub represent the respective frequency of the two alleles of said microsatellite marker in the urine DNA of said patient,

c) determining, for each microsatellite marker, the absolute value of the percentage of Allelic Imbalance (AI%) between the allele ratios (Bb/Ba) and (Ub/Ua) determined at step b), according to the following formula

AI%=((Bb/Ba)−(Ub/Ua))×100/(Bb/Ba),

and

d) comparing, for each microsatellite marker, the AI% value found for said patient with a control AI% value for the same marker found in individuals who are not predisposed nor affected with bladder cancer and wherein a difference between the patient AI% value and the control AI% value of more than a predetermined cut-off value, for at least one microsatellite marker, is indicative of a predisposition to, or of the occurrence of, a bladder cancer in said patient.

The present invention also deals with a kit for the in vitro diagnosis of a predisposition to bladder cancer or of the occurrence of a bladder cancer, wherein said kit comprises means for performing an allelotyping analysis of at least 8 microsatellite markers associated with bladder cancer which are selected from the group consisting of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

### BRIEF DESCRIPTION OF THE FIGURES

FIG. 1.: Reproducible allelic profile of independent PCRs. Seven separated PCRs were performed simultaneously from the same DNA microsatellite (D9S162) and loaded on the same gel. Allelic ratio are indicated for each analysis. The horizontal axis represents the length of the fragments as determined by size markers migrated on the same gel.

FIG. 2.: Reproducibility of the microsatellite analysis performed on paired blood and urine samples. Two independent PCRs (I and II) of selected microsatellites were performed on paired blood and urine DNAs isolated from three patients. A) For patient n^{o}1, a strong AI of 82% was observed for microsatellite D9S747 and was confirmed by a second PCR showing an AI of 85%. B) For patient n^{o}2, a weak AI of 18% observed at locus D16S310 was confirmed in a second analysis with an AI of 20%. C) For patient n^{o}3, the locus D16S310 is not altered in two independent PCRs.

FIG. 3.: Example of serial dilution of urine DNA with blood paired DNA. PCR and electrophoresis were performed as described in material and methods. When an AI of 90% is observed in urine DNA (U) as compared to blood paired DNA (B), the ¼ dilution (25U/75B) still presents a significant AI of 23%, in contrast to the 1/10 dilution which leads to no significant alteration of the allele ratio.

FIG. 4.: Sensitivity of the bladder cancer allelotyping. A) The whole population of primary bladder cancers was stratified by stages as described in Table 1. The percentages of sensitivity are calculated for AI determined with cut-offs either at 2SD (bars with vertical lines) or 3SD (bars with diagonal lines). B) The population of primary bladder cancers was stratified by grades as described in Table 1. Sensitivity is presented as in A).

The present blinded comparative study with cystoscopy and pathology diagnosis aimed to determine whether microsatellite analysis could be a valuable marker of lower urinary tract cancers, which can also be extended to the upper urinary tract cancer. In order to detect early cancer, such analysis requires high sensitivity and specificity. Using a non-isotopic and semi-automated technique, the present work focused at improving the detection of tumors in the urinary tract by the characterization of microsatellite rearrangements in urine sediment.

### DETAILED DESCRIPTION OF THE INVENTION

The inventors have now found that a comparison value between the allelic ratios of the alleles of several microsatellite markers associated with bladder cancer found respectively in the urine DNA and the blood DNA of a human individual was a reliable parameter which is highly indicative of a predisposition of the bladder cancer or of the occurrence of a bladder cancer within said individual.

More precisely, it has been shown according to the invention that the allelic ratio of a given microsatellite marker in the urine DNA and in the blood DNA from a population of individuals which are not predisposed to, nor affected by, bladder cancer is substantially constant and allows the determination of a mean control value of the allelic ratio corresponding to this population of healthy individuals, whereas the allelic ratios from the urine DNA of patients affected by bladder cancer, using the same microsatellite markers, significantly differs from the control mean value. Further, it has been found by the inventors that the allelic ratio for a given microsatellite marker found respectively in the blood DNA and in the urine DNA of the same patient also significantly differ one from each other.

Further, it has been found according to the invention that in individuals for which there is no clinical diagnosis of a bladder cancer, the imbalance between the allelic ratio of a given marker respectively in the urine DNA and in the blood DNA is indicative of a predisposition of the disease, or alternatively of a precocious stage of the disease for which no clinical diagnostic is yet available.

The results obtained by the inventors have allowed them to carry out a new in vitro method of diagnosis of a predisposition to the bladder cancer or of the occurrence of a bladder cancer in a tested individual, which takes advantage of the great reproducibility and reliability of the allelic ratio values as well as of the high statistical correlation between the value of the percentage of allelic imbalance for a given microsatellite marker in an individual and the predisposition to, or the occurrence of, a bladder cancer within said individual.

A first object of the invention consists of a method for the in vitro diagnosis of a predisposition to bladder cancer or of the occurrence of a bladder cancer in a human patient, wherein said method comprises the steps of:

a) performing an allelotyping analysis respectively (i) of the blood DNA and (ii) of the urine DNA from said patient, using a serial of microsatellite markers associated with bladder cancer,

b) determining the allele ratio for each microsatellite marker respectively (i) (Bb/Ba) for the blood DNA and (ii) (Ub/Ua) for the urine DNA, wherein:

Ba and Bb represent the respective frequency of the two alleles of said microsatellite marker in the blood DNA of said patient, and

Ua and Ub represent the respective frequency of the two alleles of said microsatellite marker in the urine DNA of said patient,

c) determining, for each microsatellite marker, the absolute value of the percentage of Allelic Imbalance (AI%) between the allele ratios (Bb/Ba) and (Ub/Ua) determined at step b), according to the following formula:

AI%=((Bb/Ba)−(Ub/Ua))×100/(Bb/Ba),

and

d) comparing, for each microsatellite marker, the AI% value found for said patient with a control AI% value for the same marker found in individuals who are not predisposed nor affected with bladder cancer and wherein a difference between the patient AI% value and the control AI% value of more than a predetermined cut-off value, for at least one microsatellite marker, is indicative of a predisposition to, or of the occurrence of, of a bladder cancer in said patient.

It has been shown according to the invention that beyond a predetermined cut-off value of the AI% value, de degree of statistical confidence was high and statistically indicative of whether the difference of the observed AI% value for a given microsatellite marker is indicative of the disease or to a predisposition to the disease.

For a high predetermined cut-off value, a high specificity of the method is reached. However, for a low predetermined cut-off value, the method can be carried out with a high sensitivity. Thus, both a high or a low predetermined cut-off value can be used at step d) of the method above, depending upon the goals which are sought.

According to a first preferred embodiment of the method above, the predetermined cut-off value used at step d) equals to (m+2SD), wherein:

m is the mean value of the variation of the allele ratio (Bb/Ba) or (Ub/Ua) found in individuals who are not predisposed nor affected with bladder cancer, and

2SD equals to twice the Standard Deviation of the mean value m.

According to a second preferred embodiment of the method above, the predetermined cut-off value used at step d) equals to (m+3SD), wherein:

m is the mean value of the variation of the allele ratio (Bb/Ba) or (Ub/Ua) found in individuals who are not predisposed nor affected with bladder cancer, and

3SD equals to three times the Standard Deviation of the mean value m.

In still another preferred embodiment of the method of the invention, the allelotyping analysis of step a) is performed by

a1) extracting DNA respectively from samples of (i) blood and (ii) urine previously collected from said patient,

a2) amplifying fragments of the extracted DNA at the respective DNA location of each microsatellite marker of the serial of satellite markers,

a3) identifying the different alleles in the amplified DNA.

For performing the allelotyping analysis as described above, the one skilled in the art may refer to the protocols which are detailed in the examples.

Generally, step a3) is performed by any conventional method of characterization of the amplified DNA.

Preferably, step a3) wherein the different alleles in the amplified DNA are identified is carried out by sequencing the amplified DNA fragments, according to any technique well known in the prior art, including that which is described in the examples.

The inventors have also found that better results are obtained when an enrichment of the urine sample in cells is performed prior the step of DNA extraction. More precisely, a better confidence in the determination of the allelic ratio of a given microsatellite marker is obtained when the cells which are initially contained in the urine sample are concentrated, or separated, or semi-purified prior the step of DNA extraction.

The enrichment of the urine samples in cells can be performed by any technique known in the art. This cell enrichment can be carried out for example by separating the various solid constituents contained in the urine samples according to their respective densities. Preferably, the separation of the nucleated cells from the other urine solid constituents, including red blood cells, is particularly sought. The cell-enrichment is preferably performed through the use of a density gradient, such as the use of an isopicnic gradient like Ficoll®.

Thus, in a preferred embodiment of the diagnostic method of the invention, the urine samples are prepared following the steps of:

collecting the urine sample from the patient,

enriching the sample in cells.

It has been found according to the invention that the minimal number of microsatellite markers to be included in the serial of microsatellite markers used in the method should be of 8 markers, which ensure to the method a sensitivity of at least 80%.

Thus, according to the method of the invention, the serial of microsatellite markers consists of at least 8 microsatellite markers associated with bladder cancer. The at least 8 microsatellite markers are selected from the group of microsatellite markers already known in the art to be associated with bladder cancer.

Preferably, the serial of microsatellite markers consists of at least 8 microsatellite markers associated with bladder cancer which are selected from the group consisting of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

In another preferred embodiment of the method above, the serial of microsatellite markers contain 17 microsatellite markers, this higher number of markers used according to the method ensuring an even better sensitivity of the diagnostic assay, as described in the examples.

Most preferably, the serial of microsatellite markers consists of the group of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

Regarding the obtained results, the present invention can be of interest not only for the diagnosis of bladder cancer but also as a therapeutic tool for the selection, the following and the modification/adaptation of a specific treatment to a given patient.

Thus, in still a further aspect, the present invention also relates to a method for monitoring, including adapting, an anti-bladder cancer therapeutic treatment administered to an individual in need of such therapeutic treatment, wherein said method comprises the steps of:

a) performing the in vitro diagnostic method of the invention which is described above in the present specification, with said patient;

b) adapting or modifying said therapeutic treatment accordingly.

Specifically, a positive therapeutic effect of the therapeutical treatment which is administered to the patient will be visualized with the in vitro diagnostic method of the invention by a decrease in the value of the AI% at one or several markers of the serial of microsatellite markers used for performing said method, which will be indicative that the therapeutical treatment is well adapted to said patient and should be pursued or, in certain cases, reduced in dosage.

In contrast, a weak therapeutic effect of the therapeutical treatment which is administered to the patient will be visualized with the in vitro diagnostic method of the invention by no change or even an increase in the value of the AI% at one or several markers of the serial of microsatellite markers used for performing said method, which will be indicative that the therapeutical treatment is, at least partially, ineffective and should be increased in dosage or alternatively replaced by another better adapted anti-cancer treatment.

The present invention also pertains to a kit which is specifically adapted for performing the bladder cancer in vitro diagnostic method of the invention, wherein said kit contains the means necessary for carrying out the allelotyping analysis of step a) of the process.

Thus, a further object of the present invention consists of a kit for the in vitro diagnosis of a predisposition to bladder cancer or of the occurrence of a bladder cancer, wherein said kit comprises means for performing an allelotyping analysis of at least 8 microsatellite markers associated with bladder cancer which are selected from the group consisting of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

In still a further aspect, the invention is also directed to a kit for the in vitro diagnosis of a predisposition to bladder cancer or of the occurrence of a bladder cancer, wherein said kit comprises means for performing an allelotyping analysis of the group consisting of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

Preferably, the means for performing the allelotyping analysis consist of pairs of nucleotide primers usable for DNA amplification at the DNA location of each of the microsatellite markers. The whole information about the nucleotide sequences of the various nucleotide primers allowing a DNA amplification at the DNA location of each of the microsatellite markers above are well known in the art. In a preferred embodiment, the one skill in the art can use the various primers described in the references cited in the examples herein.

The present invention is further illustrated by, without in no case being limited to, the examples below.

### EXAMPLES

A. Material and Methods Used in the Examples

Patients.

Urine samples and peripheral blood lymphocytes were collected from patients undergoing follow-up cystoscopy or endoscopic transurethral resection. In the blinded study performed from September 1996 to June 1998, 183 patients were included. This population comprised 136 men and 47 women (sex ratio 2.9) of 17 to 88 years old (average 62). In this study, 103 bladder cancers were analyzed, in addition to 47 cases of other malignancies, 7 cases of benign inflammatory urothelial diseases and 26 patients without malignant disease as controls. In case of suspicious bladder lesions detected by cystoscopy, the chips obtained by transurethral resection of bladder (TURB) were analyzed by the pathologist. The numbers of bladder cancer patients stratified by stage and grade are shown in the following Table 1 which reports the clinical status of the tested population.

TABLE 1

| A) Bladder cancer^{a}: | | | | |
|---|---|---|---|---|
| grade | I | II | III | 103 |
| stage pTa | 36 | 24 | 4 | 64 |
| stage pT1 | 2 | 12 | 5 | 19 |
| stage pT2 | 1 | 1 | 9 | 11 |
| stage pT3-pT4 | | 1 | 6 | 7 |
| Secondary cancer | | | | 2 |
| B) Other malignant diseases: | | | | 47 |
| Upper tract TCC^{b} | | | | 8 |
| RCC^{c} | | | | 39 |
| C) Benign urothelial diseases: | | | | 7 |
| Interstitial cystitis | | | | 2 |
| Glandular cystitis | | | | 1 |
| Prostatitis | | | | 1 |
| Pyelonephritis | | | | 1 |
| Chronic cystitis | | | | 1 |
| Inflammatory cystitis | | | | 1 |
| D) Control patients: | | | | 26 |
| PCNL^{d} | | | | 11 |
| Ureteroscopy | | | | 5 |
| Burch/Coelio Procedures | | | | 4 |
| Uretero Pelvic Junction Surgery | | | | 1 |
| Others | | | | 5 |

^{a}Bladder tumors were stratified according to the TNM UICC 1997
^{b}TCC: Transitional cell carcinoma
^{c}RCC: Renal cell carcinoma
^{d}PCNL: Percutaneous nephrolithotomy.

At the time of analysis the biologist knew neither the clinical diagnosis nor the results of pathology.

Urine and Blood DNA Extraction.

Up to 40 ml urine were collected mainly through an endoscope or a catheter. In every case, 5 ml of peripheral blood were collected on EDTA. Urine sediment cells recovered after Ficoll centrifugation were washed twice with Hanks solution and then lysed at 37° C. in 200 μl of buffer (8M urea, 2%SDS, 10 mM EDTA, 0.3M NaCl, 10 mM Tris, pH 8). After overnight digestion at 37° C. with proteinase K (200 μg/ml), followed by two phenol chloroform (1:1) treatments, precipitated DNA was dissolved in 200 μl of Tris/EDTA buffer (20 mM Tris-HCl, pH 7.6, 1 mM EDTA). For blood DNA extraction, red blood cells were disrupted in hypotonic buffer TKM1 (20 mM Tris-HCl, pH 7.6, 10 mM KCl, 10 mM MgCl₂, 2 mM EDTA) in presence of NP40 (2.5%). After centrifugation at 2,200 rpm for 10 min, pellets of leukocyte nuclei were washed once with TKM1 and then incubated overnight at 37° C. in lysis buffer (20 mM Tris-HCl, pH 7.6, 5 mM EDTA, 1% Sarkosyl, and 40 μg/ml proteinase K). DNA extraction was finally performed as described above.

Microsatellite Analysis.

Extracted DNA from each sample was amplified by PCR using previously described primers for 17 polymorphic microsatellite markers localized on chromosomes 4, 6, 8, 9, 11, 13, 14, 16, 17, 20 (27, 28) and the following table 2 reports the variations in blood/urine ratio for 26 control patients.

TABLE 2

| | | | | cut-off value (%) | |
|---|---|---|---|---|---|
| ID^{a} | Chrom. loc.^{b} | Htz. nb.^{c} | m (%)^{d} | m + 2 SD | m + 3 SD |
| D9S162 | 9p23 | 17 | 3.06 | 7 | 9 |
| IFNA | 9p22 | 17 | 4.65 | 11 | 14 |
| D16S310 | 16 | 23 | 3.61 | 11 | 14 |
| D16S476 | 16 | 25 | 3.88 | 10 | 13 |
| D4S243 | 4q32 | 21 | 3.24 | 8 | 10 |
| FGA | 4q28 | 7 | 3.07 | 8 | 11 |
| ACTBP2 | 6q13 | 26 | 5.07 | 12 | 16 |
| D9S171 | 9p21 | 24 | 4.17 | 11 | 14 |
| D9S747 | 9q | 18 | 5.56 | 14 | 18 |
| MJD52 | 14q32 | 18 | 4.11 | 11 | 14 |
| D8S307 | 8p11-21 | 11 | 3.00 | 8 | 11 |
| THO | 11 | 20 | 4.10 | 13 | 17 |
| D13S802 | 13q12 | 21 | 4.24 | 13 | 18 |
| D17S695 | 17 | 21 | 4.90 | 12 | 15 |
| D17S654 | 17 | 15 | 3.33 | 7 | 9 |
| D20S48 | 20p12 | 9 | 4.67 | 10 | 13 |
| TP53 | 17p13 | 22 | 4.32 | 10 | 13 |

^{a}ID: Genome Data Base identification.
^{b}Chrom. loc: Chromosomal location.
^{c}htz.nb: Number of informative analyzed loci
^{d}m(%): Mean of the variation of allelic ratio between urine and blood paired DNA.

PCR amplification (Omnigen Hybrid Thermocycler) were carried out in a 25 μl final volume by combining 100 ng of template, 0.6 unit of Taq polymerase in PCR buffer (1.5 mM MgCl₂, 50 mM KCl, 20 mM Tris-HCl, pH 8.4) (GIBCO-BRL Life Technology), 80 μM of each dNTP and 0.16 μM of each primer. The PCR protocol consisted in 35 cycles of 1 min at 95° C., 1 min at 50° C., 1 min at 72° C. followed by a final 5 min extension at 72° C. One primer of each pair was 5′ Cy5 labeled. Following PCR, amplified fragments were analyzed on an ALF Sequencer (Amersham-Pharmacia). This technique allows a quantitative evaluation of the allele ratio, measuring the peak height of both alleles, since it uses a unique labeling that permits accession to the raw data. Thus, it is possible to use the base line to get full sensitivity above the background.

### EXAMPLE 1

### Assay Reproducibility and Cut-Off Determination for Allelic Imbalance (AI).

As previously shown, it is possible, using a limited number of amplified microsatellites, to detect exfoliated bladder tumor cells in urine sediment (17). The allelic imbalance, defined by modification of the allele ratio in urine DNA compared to blood paired DNA depends on the percentage of tumor cells which appeared highly variable in urine pellet. The sensitivity was increased by detecting the smallest fraction of tumor cells and thereby the smallest significant allele ratio alteration by using a semi-automated fluorescent PCR sequencing analyzer.

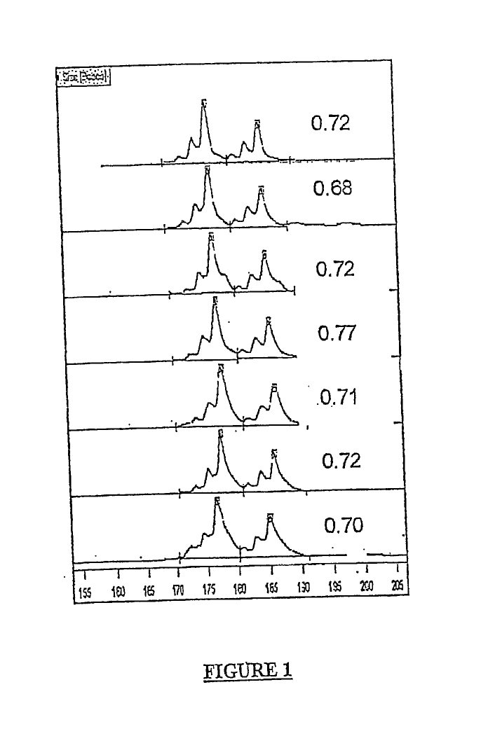

Intra-assay electrophoresis reproducibility was performed after quantification of the signal obtained for 9 simultaneous electrophoresis of the same PCR product. As an example, for the microsatellite D16S310, the CV of the allele ratio was 2.5% for blood DNA and 3% for urine DNA with a mean allele ratio of 0.89 and 0.87 respectively. Inter-assay PCR reproducibility was evaluated by measuring the CV of the allele ratio for one microsatellite fragment amplified in several independent PCRs. As shown in FIG. 1, the CV calculated from peak height measured for the microsatellite D9S162 was 3.8% with a mean allele ratio of 0.72 (n=7). By analyzing peak height variations of another microsatellite D9S747, the CV were 4.1%. These results suggested that the use of raw data and the measurement of the peak height present a good reproducibility and allowed to next determine the variability of the allele ratio between paired urine sediment and blood DNAs. The intensity of AI was calculated as a percentage (29):

AI%=absolute value ((Bb/Ba)−(Ub/Ua))×100/(Bb/Ba)

in which,

Ba and Bb represent the height of the two alleles in the blood, and

Ua and Ub represent the height of the two alleles in the urine.

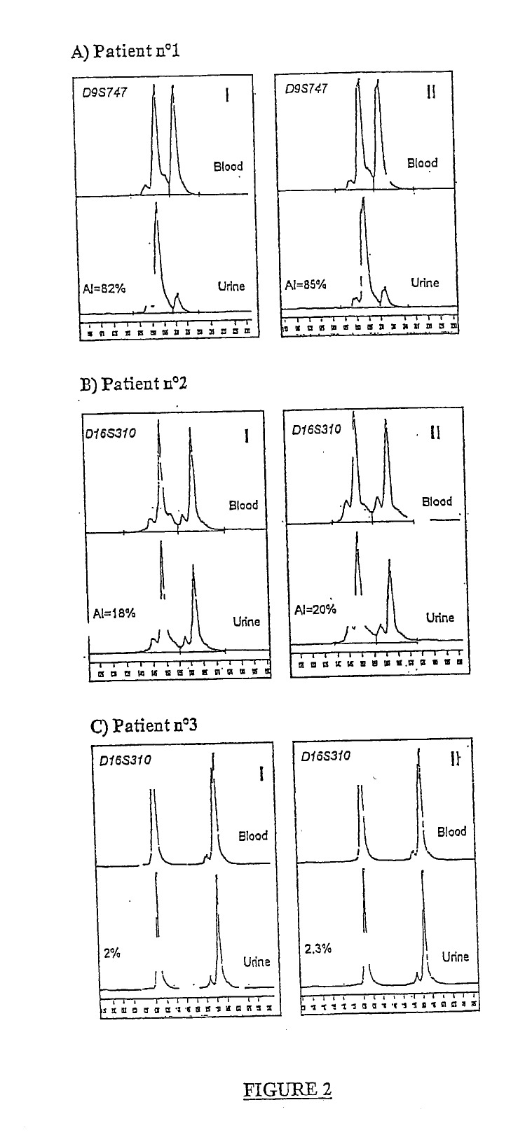

In the systematic study, the presence of an AI was confirmed by at least two independent PCRs. As expected from the inter-assay PCR results described above, an excellent reproducibility both with strong and weak AI was obtained (FIG. 2).

The cut-off value of a significant AI was determined by analyzing urine and blood DNAs of 26 control patients. When the cut-off value for each microsatellite was calculated at 2SD, a variation of these cut-offs ranging from 7% for D9S162 (n=17) to 14% for D9S747 (n=18) was observed (Table 2). At 3SD, all the normal subjects had a normal molecular analysis, but at 2SD 3 out of 26 control patients showed one significant AI leading to a specificity of 88%. However, despite this lower specificity, a cut-offs at 2SD was chosen since it results in a better sensitivity. Thus, for the molecular detection of tumor cells, it was considered indeed that it is of interest to provide the urologist with the highest sensitivity.

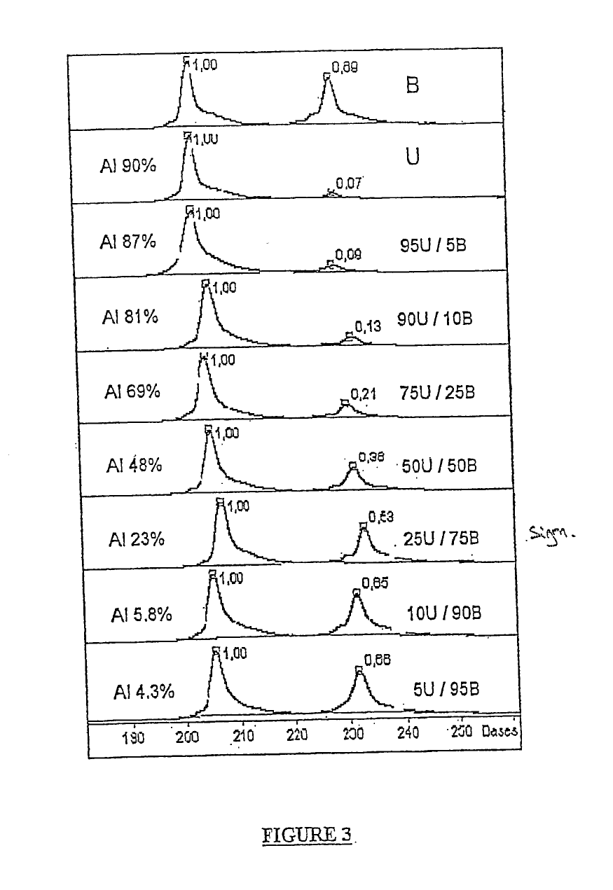

To evidence the linearity of tumor cells detection, an amplification of two highly rearranged microsatellites was performed on serial dilution of urine DNA with blood paired DNA (FIG. 3). A significant AI value was still observed with a ¼ dilution but not with a 1/10 dilution. Assuming that the percentage of AI corresponds to the percentage of tumor cells in the urine sample, experiments show that it is possible to detect an AI when urine sediment cells comprise at least 20% of tumor cells.

### EXAMPLE 2

### Results of Allelotyping and Correlation with Tumor Stage and Histological Grade.

The analysis of 17 polymorphic microsatellites was performed on paired urine sediment and control blood DNA samples and the percentage of allelic imbalance at each locus in 103 bladder cancer is reported in the following Table 3.

TABLE 3

| ID^{a} | Htz. nb.^{b} | Al %^{c} | Htz %^{d} |
|---|---|---|---|
| D9S162 | 53 | 38 | 60 |
| IFNA | 38 | 37 | 47 |
| D16S310 | 41 | 17 | 61 |
| D16S476 | 55 | 15 | 85 |
| D4S243 | 59 | 25 | 73 |
| FGA | 50 | 8 | 74 |
| ACTBP2 | 81 | 47 | 95 |
| D9S171 | 68 | 40 | 72 |
| D9S747 | 62 | 34 | 64 |
| MJD52 | 66 | 23 | 83 |
| D8S307 | 21 | 14 | 78 |
| THO | 36 | 47 | 86 |
| D13S802 | 37 | 14 | 76 |
| D17S695 | 40 | 23 | 89 |
| D17S654 | 28 | 18 | 65 |
| D20S48 | 13 | 23 | 27 |
| TP53 | 50 | 32 | 63 |

^{a}ID: Genome Data Base identification.
^{b}Htz.nb.: Number of informative analyzed loci.
^{c}Al %: Percentage of informative analyzed patients presenting an allelic imbalance at the locus.
^{d}Htz %: Percentage of analyzed patients presenting an heterozygous locus.

Except for the two markers IFNA and D20S48 that show only 47% and 27% of heterozygosity, the informativity of the other markers analyzed, ranged from 95% to 60%, confirming the ca. 70% values listed in the genome data base (GDB). Among the 183 patients at initial presentation, 129 urine specimens (70%) showed molecular anomalies with cut-off values at 2SD. AI at two or more loci was observed in 101/129 urine specimens, whereas 28 urine specimens showed AI at only one locus. No alteration was observed in 54/183 urine samples. Unlike previous studies, the presence of an additional peak corresponding to microsatellite instability was never observed (17, 30).

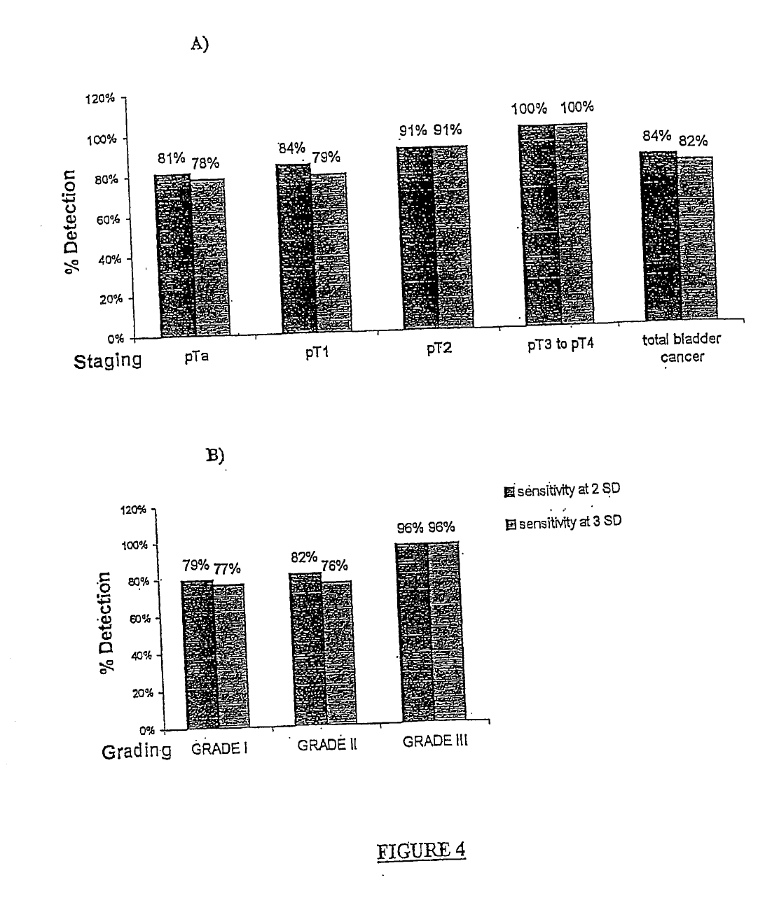

The code was then broken and clinical, pathological and molecular data compared. In all cases, the presence of bladder TCC detected by cystoscopy was confirmed by histopathological examination of tumor specimens. Among the 103 bladder TCC, AI were detected in urine specimens of 87 patients resulting in 84% sensitivity at diagnosis. Among these 87 bladder TCC with molecular anomalies, 71 urine samples showed AI at least two loci. AI at only one locus was observed in 16/87 patients. Three of these 16 patients presented weak AI values comprised between the 2SD and 3SD cut-offs, yielding 82% sensitivity at 3SD cut-off (FIG. 4A).

According to clinical stages and histological grades (TNM UICC 1997 Classification), urine specimens showed AI in 52/64 (81%) early pTa to 7/7 (100%) pT3 to pT4 stages (FIG. 4A). Taking into account the grade, the frequencies of AI increased from 79% (31/39) in grade I to 96% (23/24) in grade III bladder tumors (FIG. 4B). The two secondary bladder cancers (primaries were ovarian and vaginal malignancies) showed AI at two and three loci respectively.

The performance of each microsatellite was next analyzed in order to detect bladder cancers. All microsatellites on chromosome 9, in addition to ACTBP2 on chromosome 6q and THO on chromosome 11 were frequently altered, ranging from 34% to 47% of analyzed informative patients. Despite their high heterozygosity, four microsatellites were altered in less than 15% of analyzed cases (Table 3). From obtained data, taking into account only the 5 most frequently rearranged microsatellites, D9S162, IFNA, D9S171, D9S747 and ACTBP2, the overall sensitivity of the molecular test was still 74% (76/103) as detailed in the following Table 4.

TABLE 4

| | | Sensitivity (%)^{b} | | |
|---|---|---|---|---|
| | | 17 | 8 | 5 |
| | n^{a} | microsatellites | microsatellites | microsatellites |
| total bladder cancer | 103 | 84 | 80 | 74 |
| stage pTa | 64 | 81 | 76 | 72 |
| stage pT1 | 19 | 84 | 79 | 74 |
| stage pT2 | 11 | 91 | 82 | 82 |
| stage pT3 to pT4 | 7 | 100 | 100 | 71 |
| secondary bladder cancer | 2 | 100 | 100 | 100 |
| grade I | 39 | 79 | 74 | 74 |
| grade II | 38 | 82 | 76 | 71 |
| grade III | 24 | 96 | 92 | 75 |

^{a}n: Number of patients with bladder cancer stratified according to the TNM UICC 1997.
^{b}Sensitivities of the molecular test were determined using results obtained for all the 17 microsatellites, the 5 most frequently rearranged microsatellites (D9S162, IFNA, D9S171, D9S747, ACTBP2), or a panel of 8 microsatellites including MJD52, THO and TP53 in addition to the 5 previous markers. In all 3 cases, significant Al were determined with the 2SD cut-off values.

The addition of 3 other microsatellites, MJD52, THO and TP53, increased the overall sensitivity to 80%. Using these 8 microsatellites, 76% of pTa tumors to 100% of pT3-pT4 tumors were detected. The sensitivity calculated for each tumor grade was 74% for grade I to 92% for grade III. These results suggested that at least 9 microsatellites could be removed from the panel without significant decrease of the sensitivity.

Discussion

Sensitivity of Urine Sediment Molecular Analysis in Bladder Cancer Detection.

Several studies have shown the capacity of the microsatellite approach to reveal an urinary tract cancer through the detection of tumor cells in urine sediment (17, 28). Nevertheless, until now no comprehensive systematic study was published to analyze by a fluorescent semi-automated method, the feasibility of this molecular analysis in a significant general population of an Urology department. In the present study, it has been shown that the use of fluorescent primers allowed a highly reproducible quantitative measurement within a wide range of allele signal intensity. Consequently, it was possible to determine for each microsatellite the cut-off value for a significant rearrangement.

In the blinded study, the presence of molecular anomalies was search for in a population (183 patients) large enough to allow stratification by tumor stage and grade. In contrast to previous studies (17, 27, 30) but in agreement with other groups working with non isotopic PCR, no microsatellite instability was detected (31 and B. Grandchamp personal communication). At the 2SD cut-off, 3 out of 26 normal controls did show one significant AI. However, the possibility that these three patients (50, 55 and 76 years old respectively) with non detected malignant disease could present any occult urinary tract cancer can not be completely excluded.

Similarly, the urine samples of 5/7 patients with benign urothelial disease showed no altered microsatellite. The two other patients suffering from glandular cystitis and inflammatory bladder lesions presented two and one significant AI respectively. Both could be suspicious for cancer lesion since no alteration was observed in histological normal tissue (data not shown). It is reasonable to propose that these patients would benefit from careful follow-up. Previous studies reported similar observations (27, 30).

These results strengthen the robustness of the microsatellite analysis, since the sensitivity (84%) is in agreement with those described by several authors working with smaller populations of around 20 patients (27-31). Incomplete sensitivity could derive from two sources: first, failure of the molecular markers to detect all cases and second, inability of small tumors to exfoliate a sufficient number of cells.

In this blinded study, the cohort included patients carrying renal cell carcinoma (RCC) and upper tract TCC as controls for cancer bearing patients. 29/39 RCC and 8/8 TCC were detected, confirming recent observations (32). However, no yet defined combination of multiple modified markers can specifically identify cellular types or tumor localization. Therefore, molecular analysis should only be interpreted by the urologist with complete clinical and histological data.

Microsatellites Analysis is Effective in Detecting Early Bladder Cancers.

A similar intensity of the AI in superficial tumor and in high stage or grade tumors was observed, suggesting that most of these cancers exfoliate similarly. Interestingly, 81% (52/64) of pTa staged tumors which represent the major part of our population (64 pTa out of 103 bladder cancers) were accurately detected. Considering low-grade, sensitivity was up to 79%. These results appear to be more efficient than previously described by Shigyo et al. (31) using fluorescent PCR, but with markers located only on chromosome 9. Thus, these results strongly argue in favor of using the described semi-automated molecular technique with a 2SD cut-off. This represents significant help for both diagnosis and follow-up, since patients with pTa tumor could frequently relapse and therefore would benefit of early diagnosis. Other biochemical assays, measuring BTA (Bladder Tumor associated Antigen), NMP22 (Nuclear Matrix Protein 22), telomerase activity and FDP (Fibrin/fibrinogen Degradation Products) have been recently developed (33-40). These tests show variable sensitivities depending on the reports but they are generally less sensitive for diagnosis of early bladder cancer (stage pTa, grade I), suggesting that they would be less efficient in a routine procedure than microsatellite analysis.

Optimizing the Microsatellites Anomalies Detection.

In this study, it was also noticed that 16 bladder tumors were not diagnosed. This lack of detection could be due to a limited amount of tumor cell in the collected urines. Preliminary results showed that separation of cells through Ficoll and TRIS-Saline buffer wash were of some help in case of haematuria and urates. Most of the analyzed samples were obtained from patients who underwent TURB, thus in order to set up a non invasive test, the possibility to analyze voided urine was checked. Preliminary assays showed that first micturition of the morning would be the most enriched sample with tumor cells in comparison with day micturition or bladder wash.

The current use of 17 different microsatellite markers could be considered as irksome and expensive. However, the use of a panel of 8 microsatellites resulted only in a slight decrease of the test sensitivity from 84% to 80%. Four out of these 8 microsatellites were localized on chromosome 9, according to previous studies showing that chromosome 9 is frequently altered in early bladder cancer (11, 15 and references therein), and in agreement with allelotyping results of Shigyo et al. (31). One microsatellite was localized on chromosome 6q13. 6q has been shown to be frequently altered in haematopoietic tumors (41) as well as in ovary carcinoma and kidney cancer (42, 43), suggesting that this microsatellite would be widely use to detect cancer cells. In agreement with previous studies (44, 45), the THO marker, located on chromosome 11, is also very frequently altered in half of our population, but often associated to chromosome 9 alteration (data not shown). In contrast, MJD52 (chromosome 14q) appeared rarely associated with AI at other loci and thus was strongly informative for the presence of tumor cells. Interestingly, TP53, located in the first intron of the p53 gene, was also frequently rearranged (32%) but less than previously described, either by immunohistochemistry or by mutation scanning (12, 46, 47). This discrepancy could be explained by differences in the cohorts analyzed.

Nevertheless, this panel of 8 microsatellites should be modified to enhance the sensitivity by targeting loci which have now been shown to be frequently altered in all cancers, especially in those of bladder, kidney and prostate (15, 48, 49, 50). This includes markers on 3p (Von Hippel Lindau gene (51, 52) and TGFb receptors (53)), 5q (APC gene) (54, 55) and 10q (PTEN/MMAC1 gene) (56, 57). Furthermore, such analysis could benefit of the use of quantitative PCR (58, 59) allowing the precise targeting of genes involved in cancerogenesis and invasiveness of urinary tract cancers.

To conclude, allelotyping of urine sediment appears to be a reproducible and sensitive test for diagnosis of bladder tumors. It can be considered as a complementary tool to cystoscopy and pathology. As this test can also be efficient for the detection of tumor recurrence, the next step would be to evaluate its sensitivity versus endoscopy and cytology in routine patient follow-up. Furthermore, it would be interesting to determine whether this test could be used in the screening of selected high risk population as well as in the diagnosis of kidney cancer.

### REFERENCES

Benedict, W. F., Lerner, S. P., Zhou, J., Shen, X., Tokunaga, H., and Czerniak, B. Level of retinoblastoma protein expression correlates with p16 (MTS-1/INK4A/CDKN2) status in bladder cancer. Oncogene, 18:1197-1203, 1999.

Bodnar, A. G., Kim, N. W., Effros, R. B., and Chiu, C. P. Mechanism of telomerase induction during T cell activation. Exp. Cell Res., 228: 58-64, 1996.

Boring, C. C., Squires, T. S., Tong, T., and Montgomery, S. Cancer statistics 1994. CA Cancer J. Clin., 44: 7-26, 1994.

Bringuier, P. P., Tamimi, Y., Schuuring, E., and Schalken, J. Expression of cyclin D1 and EMS1 in bladder tumors; relationship with chromosome 11q13 amplification. Oncogene, 12:1747-1753,1996.

Bugert, P. and Kovacs, G. Molecular differential diagnosis of renal cell carcinomas by microsatellite analysis. Am. J. Pathol., 149: 2081-2088,1996.

Cairns, J. P., Chiang, P. W., Ramamoorthy, S., Kurnit, D. M., and Sidransky, D. A comparison between microsatellite and quantitative PCR analyses to detect frequent p16 copy number changes in primary bladder tumors. Clin. Cancer Res., 4: 441-444,1998.

Chiang, P-W., Song, W-J., Wu, K-Y., Korenberg, J. R., Fogel, E. J., Van Keuren, M. L., Lashkari, D., and Kurnit, D. M. Use of a fluorescent-PCR reaction to detect genomic sequence copy number and transcriptional abundance. Genome Res. 6:1013-1026,1996.

Clavel, J., Cordier, S., Boccon-Gibod, L., and Hemon, D. Tobacco and bladder cancer in males: increased risk for inhalers and smokers of black tobacco. Int. J. Cancer, 44: 605-610, 1989.

Cohen, S. C., and Johansson, S. L. Epidemiology and etiology of bladder cancer. Urologic Clinics of North America, 19: 421-428, 1992.

Czerniak, B., Chaturvedi, V., Li, L., Hodges, S., Johnston, D., Roy, J. Y., Luthra, R., Logothetis, C., Von Eschenbach, A. C., Grossman, H. B., Benedict, W. F., and Batsakis, J. G. Superimposed histologic and genetic mapping of chromosome 9 in progression of human urinary bladder neoplasia: implications for a genetic model of multistep urothelial carcinogenesis and early detection of urinary bladder cancer. Oncogene, 18: 1185-1196,1999.

D'Hallewin, M-A., and Baert, L. Initial evaluation of the bladder tumor antigen test in superficial bladder cancer. J. Urol., 155: 475-476, 1996.

Eisenberger, C. F., Schoenberg, M., Enger, C., Hortopan, S., Shah, S., Chow, N. H., Marshall, F. F., and Sidransky, D. Diagnosis of renal cancer by molecular urinalysis. J. Natl. Cancer Inst., 91: 2028-2032, 1999.

Eshleman, J. R., and Markowitz, S. D. Mismatch repair defects in human carcinogenesis. Hum. Mol. Gen., 5:1489-1494,1996.

Esrig, D., Elmajian, D., Groshens, S., Freeman, J. A., Stein, J. P., Chen, S. C., Nichols, P. W., Skinner, D. G., Jones, P. A., and Cote, R. J. Accumulation of nuclear p53 and tumor progression in bladder cancer. N. Engl. J. Med., 331: 1259-1264, 1994.

Fearon, E. R. Molecular genetic studies of the adenoma-carcinoma sequence. Adv. Intern. Med., 39:123-147,1994.

Fearon, E. R., Feinerg, A. P., Hamilton, S. H., and Vogelstein, B. Loss of genes on the short arm of chromosome 11 in bladder cancer. Nature, 318: 377-380,1985.

Giroldi, L. A., Bringuier, P-P., Shimazui, T., Jansen, K., and Schalken, J. A. Changes in cadherin-catenin complexes in the progression of human bladder carcinoma. Int. J. Cancer, 82: 70-76, 1999.

Hatta, Y., Yamada, Y., Tomonaga, M., Miyoshi, I., Said, J. W., and Koeffler, H. P. Detailed deletion mapping of the long arm of chromosome 6 in adult T-cell leukemia. Blood, 93: 613-616,1999.

Ionov, Y., Peinado, M. A., Malkhosyan, S., Shibata, D., and Perucho, M. Ubiquitous somatic mutations in simple repeated sequences reveal a new mechanism for colonic carcinogenesis. Nature, 363: 558-561, 1993.

Kallioniemi, A., Kallioniemi, O. P., Citro, G., Sauter, G., DeVries, S., Kershmann, R., Caroll, P., and Waldman, F. Identification of gains and losses of DNA sequences in primary bladder cancer by comparative genomic hybridization. Genes Chromosomes Cancer, 12:213-219,1995.

Kiemeney, L. A., Witjes, J. A., Heijbroek, R. P., Verbeek, A. L., and Debruyne, F. M. Predictability of recurrent and progressive disease in individual patients with primary superficial bladder cancer. J. Urol., 150: 60-64,1993.

Kim, I. Y., Ahn, H. J., Zelner, D. J., Shaw, J. W., Lang, S., Kato, R., Oefelein M. G., Miyazono, K., Nemeth J. A., Kozlowski, J. M., and Lee, C. Loss of expression of transforming growth factor beta type I and type II receptors correlates with tumor grade in human prostate cancer tissues. Clin. Cancer Res., 2:1255-1261,1996.

Kim, M., Katayose, Y., Li, Q., Rakkar, A. N., Li, Z., Hwang, S. G., Katayose, D., Trepel, J., Cowan, K. H., and Seth, P. Recombinant adenovirus expressing Von Hippel-Lindau-mediated cell cycle arrest is associated with the induction of cyclin-dependent kinase inhibitor p27Kip1. Biochem. Biophys. Res. Commun., 253: 672-677,1998.

Kim, N. W., Piatyszek, M. A., Prowse, K. R., Harley, C. B., West, M. D., Ho, P. L., Coviello, G. M., Wright, W. E., Weinrich, S. L., and Shay, J. W. Specific association of human telomerase activity with immortal cells and cancer. Science, 266:2011-2015,1994.

Kurth, K. H., Denis, L., Bouffioux, C., Sylvester, R., Debruyne, F. M., Pavone-Macaluso, M., and Oosterlinck, W. Factors affecting recurrence and progression in superficial bladder tumors. Eur. J. Cancer, 31A: 1840-1846, 1995.

Landman, J., Chang, Y., Kavaler, E., Droller, M. J., and Liu, B. C. Sensitivity and specificity of NMP-22, telomerase, and BTA in the detection of human bladder cancer. Urology, 52: 398-402, 1998.

Lee, J. H., Kavanagh, J. J., Wildrick, D. M., Wharton, J. T., and Blick, M. Frequent loss of heterozygosity on chromosomes 6q, 11, and 17 in human ovarian carcinomas. Cancer Res. 50: 2724-2728, 1990.

Leyh, H., Marberger, M., Conort, P., Sternberg, C., Pansadoro, V., Pagano, F., Bassi, P., Boccon-Gibod, L., Ravery, V., Treiber, U., and Ishak, L. Comparison of the BTA stat test with voided urine cytology and bladder wash cytology in the diagnosis and monitoring of bladder cancer. Eur. Urol., 35: 52-56, 1999.

Liu, B., Nicolaides, N. C., Markowitz, S., Willson, J. K. V., Parsons, R. E., Jen, J., Papadopolous, N., Peltomäki, P., de la Chapelle, A., Hamilton, S. R., Kinzler, K. W., and Vogelstein, B. Mismatch repair gene defects in sporadic colorectal cancers with microsatellite instability. Nature Genet., 9: 48-55, 1995.

Loeb, L. A. Microsatellite instability: marker of a mutator phenotype in cancer. Cancer Res., 54: 5059-5063,1994.

Lubensky, I. A., Gnarra, J. R., Bertheau, P., Walther, M. M., Linehan, W. M., and Zhuang, Z. Allelic deletions of the VHL gene detected in multiple microscopic clear cell renal lesions in von Hippel-Lindau disease patients. Am. J. Pathol., 149: 2089-2094,1996.

Mao, L., Lee, D. J., Tockman, M. S., Erozan, Y. S., Askin, F., and Sidransky, D. Microsatellite alterations as clonal markers for the detection of human cancer. Proc. Natl. Acad. Sci. USA, 91: 9871-9875,1994.

Mao, L., Schoenberg, M. P., Scicchitano, M., Erozan, Y. S., Merlo, A., Schwab, D., and Sidransky, D. Molecular detection of primary bladder cancer by microsatellite analysis. Science, 271: 659-662,1996.

Miyamoto, H., Kubota, Y., Shuin, T., Torigoe, S., Hosaka, M., Iwasaki, Y., Danenberg, K., and Danenberg, P. V. Analyses of p53 gene mutations in primary human bladder cancer. Oncol. Res., 5: 245-249,1993.

Morita, R., Saito, S., Ishikawa, J., Ogawa, O., Yoshida, O., Yamakawa, K., and Nakamura, Y. Common regions of deletion on chromosomes 5q, 6q, and 10q in renal cell carcinoma. Cancer Res. 51: 5817-5820,1991.

Mourah, S., Cussenot, O., Vimont, V., Desgrandchamps, F., Teillac, P., Cochant-Priollet, B., Le Duc, A., Fiet, J., and Soliman, H. Assessment of microsatellite instability in urine in the detection of transitional-cell carcinoma of the bladder. Int. J. Cancer, 79: 629-633,1998.

Murphy, W. M., Soloway, M. S., Jukkola, A. F., Crabtree, W. N., and Ford, K. S. Urinary cytology and bladder cancer. The cellular features of transitional cell neoplasms. Cancer, 53: 1555-1565, 1984.

Orntoft, T. F., and Wolf, H. Molecular alterations in bladder cancer. Urol. Res., 26: 223-233,1998.

Özen, H. Bladder Cancer. Curr. Opin. Oncol., 10: 273-278,1998.

Peltomäki, P., Aaltonen, L. A., Sistonen, P., Pylkkänen, L., Mecklin, J-P., Järvinen, H., Green, J. S., Jass, J. R., Weber, J. L., Leach, F. S., Petersen G. M., Hamilton, S. R., de la Chapelle, A., and Vogelstein, B. Genetic mapping of a locus predisposing to human colorectal cancer. Science, 260: 810-812,1993.

Ramakumar, S., Bhuiyan, J., Besse, J. A., Roberts, S. G., Wollan, P. C., Blute, M. L. and O'Kane, D. J. Comparison of screening methods in the detection of bladder cancer. J. Urol., 161: 388-394,1999.

Reznikoff, C. A., Belair, C. D., Yeager, T. R., Savelieva, E., Blelloch, R. H., Puthenveettil, J. A., and Cuthill, S. A molecular genetic model of human bladder cancer pathogenesis. Semin. Oncol., 23: 571-584,1996.

Richter, J., Jiang, F., Gorog, J. P., Sartorius, G., Egenter, C., Gasser, T. C., Moch, H., Mihatsch, M. J., and Sauter, G. Marked genetic differences between stage pTa and stage pT1 papillary bladder cancer detected by comparative genomic hybridization. Cancer Res., 57: 2860-2864, 1997.

Sanchez De La Muela, P., Rosell, D., Agüera, L., De Castro, F., Isa, W., Robles, J. E., Zudaire, J. J., and Berian, J. M. Superficial bladder cancer: survival and pronostic factors. Eur. Urol., 20: 184-191, 1991.

Saran, K. K., Gould, D., Godec, C. J., and Verma, R. S. Genetics of bladder cancer. J. Mol. Med., 74:441-445,1996.

Sarosdy, M. F. The use of the BTA test in the detection of persistent or recurrent transitional-cell cancer of the bladder. World J. Urol., 15: 103-106, 1997.

Shigyo, M., Sugano, K., Fukayama, N., Taniguchi, T., Tobisu, K-I., Fujimoto, H., Doi, N., Niwakawa, M., Shimamura, K., Tsukamoto, T., Sekiya, T., and Kakizoe, T. Allelic loss on chromosome 9 in bladder cancer tissues and urine samples detected by blunt-end single-strand DNA conformation polymorphism. Int. J. Cancer, 78: 425-429, 1998.

Sidransky, D., Von Eschenbach, A., Tsai, Y. C., Jones, P., Summerhayes, I., Marshall, F., Paul, M., Green, P., Hamilton, S. R., Frost, P., and Vogelstein, B. Identification of p53 gene mutations in bladder cancers an urine samples. Science, 252: 706-709,1991.

Steiner, G., Schoenberg, M. P., Jurgen, F. L., Mao, L., and Sidransky, D. Detection of bladder cancer recurrence by microsatellite analysis of urine. Nat. Med., 3: 621-624,1997.

Sugano, K., Nakashima, Y., Yamaguchi., K., Fukayama., N., Maekawa, M., Ohkura, H., Kakizoe, T., and Sekiya, T. Sensitive detection of loss of heterozygosity in the Tp53 gene in pancreatic adenocarcinoma by fluorescence-based single-strand conformation polymorphism analysis using blunt-end DNA fragments. Genes Chromosomes Cancer, 15: 157-164, 1996.

Suzuki, H., Freije, D., Nusskern, D. R., Okami, K., Cairns, P., Sidransky, D., Isaacs, W. B., and Bova, G. S. Interfocal heterogeneity of PTEN/MMAC1 gene alterations in multiple metastatic prostate cancer tissues. Cancer Res., 58: 204-209,1998.

Thibodeau, S. N., Bren, G., and Schaid, D. Microsatellite instability in cancer of the proximal colon. Science, 260: 816-819, 1993.

Thibodeau, S. N., French, A. J., Cunningham, J. M., Tester, D., Burgart, L. J., Roche, P. C., Mc Donnel, S. K., Schaid, D. J., Walsh Vockley, C., Michels, V. V., Farr Jr., G. H., and O'Connell, M. J. Microsatellite Instability in colorectal cancer: different mutator phenotypes and the principal involvement of HMLH1. Cancer Res., 58:1713-1718,1998.

Thomas, L., Leyh, H., Marberger, M., Bombardieri, E., Bassi, P., Pagano, F., Pansadoro, V., Sternberg, C. N., Boccon-Gibod, L., Ravery, V., Le Guludec, D., Meulemans, A., Conort, P., and Ishak, L. Multicenter trial of the quantitative BTA TRAK assay in the detection of bladder cancer. Clin. Chem., 45: 472-477,1999.

Vet, J. A., Bringuier, P. P., Poddighe, P. J., Karthaus, H. F., Debruyne, F. M., and Schalken, J. A. p53 mutations have no additional prognostic value over stage in bladder cancer. Br. J. Cancer, 70: 496-500,1994.

Voeller, H. J., Truica, C. I., and Gelmann, E. P. β-Catenin mutations in human prostate cancer. Cancer Res., 58: 2520-2523, 1998.

Vogelstein, B., Fearon, E. R., Kern, S. E., Hamilton, S. R., Preisinger, A. C., Nakamura, Y., and White, R. Allelotype of colorectal cancer. Science, 244: 207-210, 1989.

Watanabe, M., Kakiuchi, H., Kato, H., Shiraishi, T., Yatani, R., Sugimura, T., and Nagao, M. APC gene mutations in human prostate cancer. Jpn. J. Clin. Oncol., 26: 77-81,1996.

Zlotta, A. R., Noel, J. C., Fayt, I., Drowart, A., Van Vooren, J. P., Huygen, K., Simon, J., and Schulman, C. C. Correlation and prognostic significance of p53, p21WAF1/CIP1 and Ki-67 expression in patients with superficial bladder tumors treated with bacillus Calmette-Guerin intravesical therapy. J. Urol., 161: 792-798,1999.

What is claimed is:

1. A method for the in vitro diagnosis of a predisposition to bladder cancer or of the occurrence of a bladder cancer in a human patient, wherein said method comprises the steps of:
a) performing an allelotyping analysis respectively (i) of the blood DNA and (ii) of the urine DNA from said patient, using a serial of microsatellite markers associated with bladder cancer,
b) determining the allele ratio for each microsatellite marker respectively (i) (Bb/Ba) for the blood DNA and (ii) (Ub/Ua) for the urine DNA, wherein:
Ba and Bb represent the respective frequency of the two alleles of said microsatellite marker in the blood DNA of said patient, and
Ua and Ub represent the respective frequency of the two alleles of said microsatellite marker in the urine DNA of said patient,
c) determining, for each microsatellite marker, the absolute value of the percentage of Allelic Imbalance (AI%) between the allele ratios (Bb/Ba) and (Ub/Ua) determined at step b), according to the following formula:
AI%=((Bb/Ba)−(Ub/Ua))×100/(Bb/Ba),
and
d) comparing, for each microsatellite marker, the AI% value found for said patient with a control AI% value for the same marker found in individuals who are not predisposed nor affected with bladder cancer and wherein a difference between the patient AI% value and the control AI% value of more than a predetermined cut-off value, for at least one microsatellite marker, is indicative of a predisposition to, or of the occurrence of, a bladder cancer in said patient.

2. The method of claim 1, wherein the predetermined cut-off value used at step d) equals to (m+2SD), wherein:
m is the mean value of the variation of the allele ratio (Bb/Ba) or (Ub/Ua) found in individuals who are not predisposed nor affected with bladder cancer, and
2SD equals twice the Standard Deviation of the mean value m.

3. The method of claim 1, wherein the predetermined cut-off value used at step d) equals to (m+3SD), wherein
m is the mean value of the variation of the allele ratio (Bb/Ba) or (Ub/Ua) found in individuals who are not predisposed nor affected with bladder cancer, and
3SD equals three times the Standard Deviation of the mean value m.

4. The method of claim 1, wherein the allelotyping analysis of step a) is performed by
a1) extracting DNA respectively from samples of (i) blood and (ii) urine previously collected from said patient,
a2) amplifying fragments of the extracted DNA at the respective DNA location of each microsatellite marker of the serial of satellite markers,
a3) identifying the different alleles in the amplified DNA.

5. The method of claim 4, wherein step a3) is performed by sequencing the amplified fragments.

6. The method of claim 4, wherein the urine samples are prepared following the steps of:
collecting the urine sample from the patient,
enriching the sample in cells.

7. The method of any one of claims 1 to 6, wherein the serial of microsatellite markers consists of at least 8 microsatellite markers associated with bladder cancer.

8. The method of any one of claims 1 to 6, wherein the serial of microsatellite markers consists of at least 8 microsatellite markers associated with bladder cancer which are selected from the group consisting of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

9. The method of any one of claims 1 to 6, wherein the serial of microsatellite markers consists of the group of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

10. A method for monitoring, including adapting, an anti-bladder cancer therapeutic treatment administered to an individual in need of such therapeutic treatment, wherein said method comprises the steps of:
a) performing the in vitro diagnostic method of claim 1 with said individual;
b) adapting, modifying or replacing said therapeutic treatment, accordingly.

11. A kit for the in vitro diagnosis of a predisposition to bladder cancer or of the occurrence of a bladder cancer, wherein said kit comprises means for performing an allelotyping analysis of at least 8 microsatellite markers associated with bladder cancer which are selected from the group consisting of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

12. A kit for the in vitro diagnosis of a predisposition to bladder cancer or of the occurrence of a bladder cancer, wherein said kit comprises means for performing an allelotyping analysis of the group consisting of the following microsatellite markers D9S162, IFNA, D16S310, D16S476, D4S243, FGA, ACTBP2, D9S171, D9S747, MJD52, D8S307, THO, D13S802, D17S695, D17S654, D20S48, TP53.

13. The kit of any one of claims 11 and 12, wherein the means for performing the allelotyping analysis consist of pairs of nucleotide primers usable for DNA amplification at the DNA location of each of the microsatellite markers.
